(19) United States
(12) Patent Application Publication
(10) Pub. No.: US 2011/0098366 A1
(43) Pub. Date: Apr. 28, 2011

(54) Pharmaceutical Preparation in Containers that are Pervious to Water Vapor and Have Improved Stability

(76) Inventors: Rolf Groteluschen, Aukrug (DE); Henning Ueck, Bekmunde (DE); Thomas Zimmeck, Hohenlockstedt (DE)

(21) Appl. No.: 12/997,909

(22) PCT Filed: May 30, 2009
(86) PCT No.: PCT/EP09/03894

§ 371 Date: Dec. 14, 2010

(30) Foreign Application Priority Data
Jun. 16, 2008 (DE) 202008008079.7

(51) Int. Cl. A61K 47/12

(52) U.S. Cl. 514/784

(57) ABSTRACT

The present invention relates to a pharmaceutical preparation, comprising a mixture of at least one pharmaceutical active ingredient, at least one alcohol component and at least one triglyceride component, in a plastic container, characterized in that the plastic has a water vapour permeability of no more than 3.0 g/m²/24 h (measured on a film 100 μm thick at 40° c. and 90% relative humidity.

The present invention relates to liquid or semisolid, non-aqueous pharmaceutical preparations comprising alcohols and triglycerides as carrier substances in containers pervious to water vapor. In particular, the invention relates to pharmaceutical preparations containing nitroglycerin as the active ingredient, which are contained in a plastic bottle.

Excipients for pharmaceutical applications must meet high requirements. In addition to technological reasonableness and suitability for the purpose, safety is an important criterion in the selection of such a substance. Alcohols and triglycerides are commonly used excipients for nonaqueous preparations. Frequently used alcohols include monovalent alcohols such as ethanol, 1- and 2-propanol, and butanol, but also bivalent and polyvalent alcohols such as propylene glycol or glycerol. Commonly used triglycerides include natural oils and fats as well as semisynthetic products such as medium-chain triglycerides, which, for example, are sold under the trade name Miglyol® 812.

relatively inert with regard to interactions with the fill material; it is transparent and protects the contents from outside influences. However, it also has disadvantages, in particular the lack of break resistance and its considerably higher weight in comparison to plastic. These disadvantages are particularly evident with pharmaceuticals that patients take with them when travelling or constantly carry on their person. The lack of break resistance is particularly problematic because they pharmaceutical package with the glass bottle or glass ampoule may easily slip out of the patient's hand in an emergency situation.

Alternatives, principally containers made of plastic, are conceivable. They are in any case lighter than glass bottles. However, other criteria such as break resistance, transparency, and inertness in relation to the content substances must be met, which limits the selection of potential plastic materials.

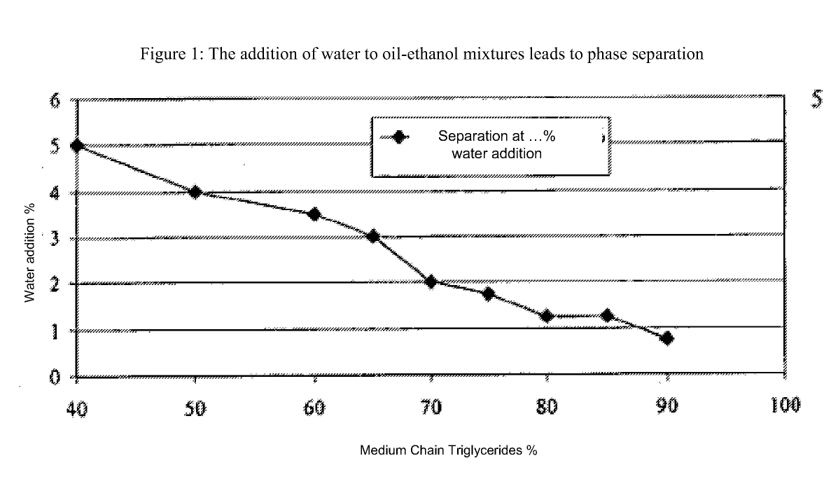

In the context of studies upon which the present invention is based, it became evident that another characteristic is of great importance: oil/alcohol preparations absorb humidity in the form of water vapor from the ambient air. Because of the affinity of water for alcohol and the hydrophobic, i.e., water-repellent, properties of oil, separation of the oil and alcohol components of the preparation may occur above a certain water content. In the process, the alcohol component together with the absorbed water form a first phase, and the oil (with a residual quantity of the alcohol, if any) a second phase. The maximum quantity of water that can be absorbed before phase separation results depends on the type of alcohol and oil components as well as the ratio of the quantities. More water can be absorbed at higher alcohol concentrations before resulting in separation. For example, limiting concentrations for mixtures comprising ethanol and medium-chain triglycerides (Miglyol® 812) that lead to separation are shown in FIG. 1. The values lacking for 100% on the X-axis correspond to the ethanol content of each particular solution used.

International guidelines generally require that drugs be stable for six months at 40° C. and 75% relative humidity and—depending on climatic zone—over their entire shelf life at 30° C. and 65% relative humidity or at 25° C. and 60% relative humidity, as the case may be. For logistical and marketing reasons, a shelf life of at least 2 years, preferably a shelf life of 3 years is generally aimed for. However, this shelf life is often not achieved with oil/alcohol preparations. There is therefore a need for products in plastic packages that meet these requirements.

The task of the present invention is therefore to provide a pharmaceutical preparation that is stable in storage comprising a mixture of at least one alcohol component and at least one triglyceride component in a plastic container.

This problem is solved by the characteristics of the independent claims. The dependent claims define advantageous embodiments of the invention.

It has surprisingly been found that plastic materials with a perviousness to water vapor of not more than 3.0 g/m2/24h are suitable as containers for pharmaceutical preparations which have a tendency towards water-induced separation resulting from their content of triglycerides and alcohol. The preparations according to the invention are stable when stored for at least 6 months at 40° C. and 75% relative humidity.

The pharmaceutical preparation is preferably a liquid or semisolid preparation, particularly preferably a sprayable liquid preparation. The preparation comprises an alcohol component, a triglyceride component, and an active pharmaceutical ingredient which is preferably soluble in the preparation.

The alcohol component is preferably selected from a group consisting of ethanol, 1-propanol, 2-propanol, and propylene glycol, whereby ethanol is particularly preferred. The preferred ethanol content in the preparations according to the invention is between 10% and 80% per weight in relation to the overall weight of the preparation.

The triglyceride component is preferably selected from the group of plant and animal fats and oils. Particularly preferred are medium-chain triglycerides, which are preferably contained in the preparations according to the invention in a quantity of between 10% and 80% by weight in relation to the overall weight of the preparations according to the invention.

The active ingredient contained in the preparations according to the invention is preferably glycerol trinitrate (GTN). It is preferably contained in the preparations according to the invention in a quantity of 0.2 to 5% by weight in relation to the overall weight of the preparation.

Preferred materials for the plastic container of the preparations according to the invention include cycloolefin polymers and cycloolefin copolymers.

The following examples explain the invention in greater detail.

### EXAMPLE 1

### Packaging in Bottles Made of Polyethylene Terephthalate (PET), Polycarbonate (PC), and Polypropylene (PP)

In this example, a drug solution comprising the active ingredient GTN, which according to prior art is on the market in a glass bottle, is tested for storage stability in plastic bottles.

For 1 kg of drug solution, 607 g medium-chain triglycerides, 7.2 g peppermint oil, 200 g ethanol abs., 20 g medium-chain partial glycerides, and 166 g glycerol trinitrate (GTN) are weighed and thoroughly mixed in medium-chain triglycerides (as a 5% solution, containing 8.3 g GTN). The solution is filled in amounts of 14.9 g into 20-ml plastic bottles made of various materials (PET [polyethylene terephthalate], PC [polycarbonate], and PP [polypropylene]), closed with a spray pump, weighed individually, and stored under the following storage conditions: 25° C./60% relative humidity; 30° C./60% relative humidity, and 40° C/75% relative humidity. The bottles are again weighed after one and three months.

The following table shows the weight changes of the individual bottle types:

| | | Storage condition | | |
|---|---|---|---|---|
| Material | Time of testing | 25° C./60% | 30° C./60% | 40° C./75% |
| PET | 1 month | 0.04 g | 0.06 g | 0.11 g |
| | 3 months | 0.11 g | 0.13 g | 0.22 g |
| PC | 1 month | 0.10 g | 0.12 g | 0.19 g |
| | 3 months | 0.19 g | 0.22 g | 0.31 g* |
| PP | 1 month | <0.01 g | <0.01 g | −0.01 g¹⁾ |
| | 3 months | 0.01 g | 0.01 g | −0.01 g¹⁾ |

*Phase separation (water content: 2.6%)
¹⁾Weight loss resulting from loss of ethanol from the spray solution

The weight change corresponds in first approximation to the water absorption from the ambient air. This is dependent on the relative humidity of the air. In polycarbonate, the critical limit is exceeded after only three months, and phase separation occurs. Bottles made of PET absorb approximately 30-50% less water from the ambient air. In polypropylene bottles stored at 40° C./75% humidity, the weight gain from water is balanced by the loss of ethanol so that the absorption of water cannot be determined by weighing alone.

This example shows that polycarbonate is not suitable as a packaging material. In further testing, water absorption is not determined by weighing the difference but by semi-micro titration.

### EXAMPLE 2

### Comparison of One Preparation Containing Active Ingredient and One Containing No Active Ingredient in PET Bottles

For 1 kg of preparation containing no active ingredient, 773 g of medium- chained triglycerides, 7.2 g peppermint oil, 200 g ethanol abs., And 20 g medium-chained partial glycerides are weighed and thoroughly mixed. The solution is filled in amounts of 14.9 g into 20-ml plastic bottles made of PET, closed with a spray pump, and stored at 40° C./75% relative humidity. Analogously, bottles with a preparation containing active ingredient are filled and stored in accordance with Example 1.

The water content is determined using the semi-micro method in the European Pharmacopeia, using a 787 KF Titrino (Metrohm Corp.). Before beginning the measurement, the sample is titrated in a titration vessel and the drift is determined. Then 1.0 to 1.5 g test solution is introduced with a syringe into the titration vessel. The weight is determined by back weighing the syringe. The result is expressed as a percentage after titrating the sample.

The following table shows each water content after storage at 40° C. and 75% relative humidity:

| Water content after storage period of | 4 weeks | 6 weeks | 8 weeks |
|---|---|---|---|
| Preparation without active ingredient | 0.80% | | 1.24% |
| Preparation containing active ingredient | | 1.27% | 1.41% |

Phase separation occurs after eight weeks of storage at an elevated atmospheric humidity. As a result, bottles made of PET are not suitable.

### EXAMPLE 3

### Comparison of Other Plastic Materials in a Stress Test at 40° C. and 75% Relative Humidity

The solution according to Example 1 is filled into the bottles having the following dimensions and materials, closed, and stored at 40° C./75% relative humidity.

| Material | Nominal volume | Lid | Fill quantity |
|---|---|---|---|
| Polyamide | 10 ml | Screw cap | 6 g |
| Cycloolefin copolymer | 10 ml | Screw cap | 6 g |
| (Topas ®) | | | |
| Polypropylene | 10 ml | Screw cap | 12 g |

The water content is determined at various times according to the method described in Example 2. The results obtained are summarized in the following table.

| Water content after storage period of | 1 month | 3 months | 6 months |
|---|---|---|---|
| Polyamide | 0.90% | * | |
| Cycloolefin copolymer | 0.26% | 0.51% | 0.84% |
| (Topas ®) | | | |
| Polypropylene | 0.31% | 0.62% | 0.94% |

* The experiment with polyamide bottles was terminated after one month because it was already clear at this point that the water content would rise above 1% within 6 months.

This experiment shows that bottles made of polyamide do not meet these requirements either. Bottles made of cycloolefin copolymer (Topas®) are particularly suitable because they absorb less water than do bottles made of polypropylene in spite of their less advantageous surface-to-volume ratio.

### EXAMPLE 4

### Storage in Bottles Made of Cycloolefin Polymer at Various Temperatures

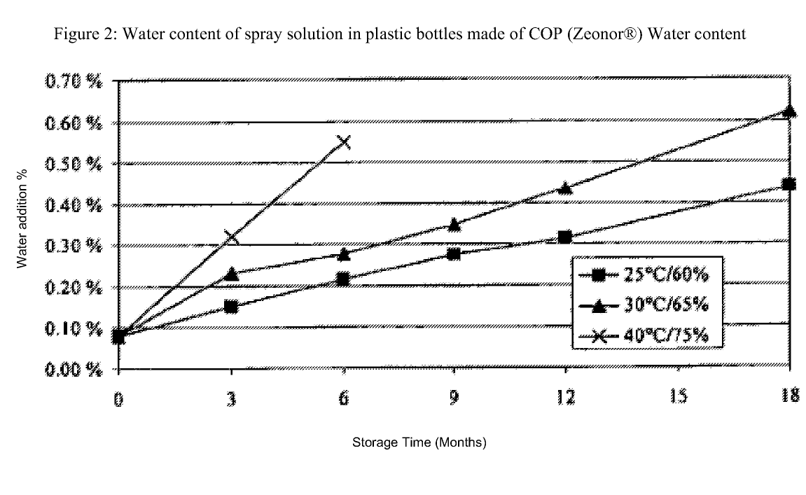

The solution according to Example 1 is filled in amounts of 12 g each into 20-ml bottles made of cycloolefin polymer (Zeonor®), closed with a spray pump, and stored at 25° C./60% relative humidity, at 30° C./60% relative humidity, and at 40° C./75% relative humidity. The water content is determined at three-month intervals using the method described in Example 2. The results are shown in FIG. 2.

Because water absorption occurs in a manner approaching linearity, it may be assumed that the water content of the preparation does not exceed the critical value of 1% within 24 months at 30° C./65% relative humidity or within 36 months at 25° C./60% relative humidity.

### EXAMPLE 5

To produce 1 kg of a drug solution, 607 g medium-chain triglycerides, 7.2 g peppermint oil, 200 g ethanol abs., 20 g medium-chain partial glycerides, and 166 g glycerol trinitrate (GTN) are weighed and thoroughly mixed in medium-chaintriglycerides (as a 5% solution, containing 8.3 g GTN). The solution is filled in amounts of 14.9 g into bottles made of cycloolefin polymer (Zeonor®), and the bottles are closed with a spray pump. The compound was stable in the bottles stored over 6 months at 40° C. and 75% relative humidity.

1-7. (canceled)

8. A stabilized pharmaceutical preparation comprising:
a pharmaceutical composition comprising components which exhibit water-induced separation when exposed to water; and
a container comprising a plastic having a perviousness to water of not more than 3.0 g/m²/day, wherein said pharmaceutical composition is stabilized when stored in said container.

9. The pharmaceutical preparation of claim 8, wherein the plastic comprises a cycloolefin polymer (COP).

10. The pharmaceutical preparation of claim 8, wherein the plastic comprises a cycloolefin copolymer (COC).

11. The pharmaceutical preparation of claim 8, wherein the pharmaceutical composition comprises at least one alcohol component.

12. The pharmaceutical preparation of claim 11, wherein the alcohol is selected from the group consisting of ethanol, 1-propanol, 2-propanol, and propylene glycol.

13. The pharmaceutical preparation of claim 8, wherein the pharmaceutical composition comprises at least one triglyceride component.

14. The pharmaceutical preparation of claim 13, wherein the at least one triglyceride component is selected from the group consisting of plant fats and oil, and animal fats and oils.

15. The pharmaceutical preparation of claim 13, wherein the at least one triglyceride component comprises a medium-chained triglyceride.

16. The pharmaceutical preparation of claim 8, wherein the components comprise a mixture of at least one active pharmaceutical ingredient, at least one alcohol component, and at least one triglyceride.

17. The pharmaceutical preparation of claim 16, wherein the pharmaceutical composition comprises a mixture of components of 10 to 80% ethanol by weight, 10 to 80% triglycerides by weight, and 0.2 to 5% glyceryl trinitrate by weight, in each case in relation to the overall weight of the mixture.

18. The pharmaceutical preparation of claim 8, wherein the perviousness to water of not more than 3.0 g/m²/day is measured using film having a thickness of 100 μm at 40° C. and 90% relative humidity.

19. A method of reducing the absorption of water by a pharmaceutical composition comprising:
placing said pharmaceutical composition in a container comprising plastic having a perviousness to water of not more than 3.0 g/m²/day;
wherein said pharmaceutical composition comprises components which exhibit water-induced separation when exposed to water.

20. The method of claim 19, wherein the pharmaceutical composition is maintained at 40° C. and 75% relative humidity for six months and has less than 1% water content after six months.

21. The method of claim 19, wherein the pharmaceutical composition, when maintained at 30° C. and 65% relative humidity for 24 months and has less than 1% water content after 24 months.

22. The method of claim 19, wherein the pharmaceutical composition is maintained at 25° C. and 60% relative humidity for 36 months and has less than 1% water content after 36 months.

23. The method of claim 19, wherein the pharmaceutical composition is a liquid or semisolid composition.

24. The method of claim 23, wherein the liquid composition is sprayable.

25. The method of claim 19, wherein the pharmaceutical composition comprises an alcohol, a triglylceride, and an active pharmaceutical ingredient.

26. The method of claim 25, wherein the alcohol is selected from the group consisting of ethanol, 1-propanol, 2-propanol, and propylene glycol.

27. The method of claim 26, wherein the pharmaceutical composition comprises 10-80% by weight of alcohol.

28. The method of claim 25, wherein the triglyceride is selected from the group consisting of plant fats and oils, and animal fats and oils.

29. The method of claim 25, wherein triglyceride is a medium chain triglyceride.

30. The method of claim 25, wherein the pharmaceutical composition comprises 10-80% by weight of triglyceride.

31. The method of claim 25, wherein the active pharmaceutical ingredient is glycerol trinitrate.

32. The method of claim 25, wherein the pharmaceutical composition comprises 0.2-5% by weight of active pharmaceutical ingredient.

33. The method of claim 19, wherein the container comprises a cycloolefin copolymer (COP) or a cycloolefin copolymer (COC).

34. The method of claim 19, wherein the enclosed container has a screw cap lid.

35. The method of claim 19, wherein the screw cap lid includes a spray pump.
